(19) United States
(12) Patent Application Publication
(10) Pub. No.: US 2005/0089537 A1
(43) Pub. Date: Apr. 28, 2005

(54) Personal lubricant with silicone polymers

(76) Inventor: Jason C. Birnholz, Highland Park, IL (US)

Correspondence Address: Charles E. Temko, 22 Marion Road, Westport, CT, 06880, US

(21) Appl. No.: 10/693,711

(22) Filed: Oct. 27, 2003

(51) Int. Cl. A61K 9/14

(52) U.S. Cl. 424/401

(57) ABSTRACT

The invention relates to viscous silicone (organo-polysiloxane) fluids comprising a volatile silicone or hydrocarbon and one or more silicone polymers and their use as personal lubricants alone or with oil soluble substances conferring trans or intradermal functionality.

### RELATED APPLICATION

Reference is made to my copending provisional application Ser. No. 60/406,071 filed Aug. 27, 2002, to which a claim of priority is made.

### BACKGROUND OF THE INVENTION

Personal lubricants have many common uses such as prevention or lessening chafing of skin rubbing against skin, cloth, leather, plastics, and other materials, facilitating introduction of instruments into body cavities, including thermometers, catheters, specula, imaging and telemetry devices, and easing on surgical gloves, condoms, prosthetic supports, thermal insulating suits and similar garments and devices. Personal lubricants have been used since antiquity to facilitate sexual relations, especially when there is vaginal dryness, condom use, and when diminished penile rigidity or increased vaginal muscle tone make difficult or preclude entry and movement.

Olive oil seems to have been promoted as a sexual lubricant at least as early as Roman times. Petrolatum (Vaseline) fulfilled this role for much of the twentieth century, being largely replaced by water soluble, lubricous gels during the last 25 years. A few commercial personal lubricants contain dimethicone or dimethicone copolymer and water resulting in a slipper liquid that is difficult to apply because of its very low viscosity.

It is known in the cosmetic arts to incorporate an organopolysilocone elastomer in a hydrocarbon vehicle which evaporates to provide a smooth powder-like feel to the skin which readily receives cosmetics, as described in U.S. Pat. No. 6,387,405.

It is also known to employ silicone gels with salicylic acid for the treatment of skin problems such as acne and siborrea, as described in U.S. Pat. No. 6,384,023. In such applications, the salicylic acid serves as a solvent for the excess sebum, thereby removing the principal cause of the dermal condition.

However, to date, the use of viscous silicone fluids in combination with a volatile silicone vehicle, as a personal internal lubricant, has not been appreciated. The result of such use is the provision of a lubricant which lacks the greasy or oily consistency encountered with the use of prior art lubricants, and which will remain in situ where applied to provide a dry buy slippery feeling.

I have found that a combination of a volatile, low molecular weight siloxane and a polymerized or gelled polysiloxane elastomer yield a viscous gel (greater than 50,000 cp) that goes on “dry”, depositing a long-lasting, velvety smooth surface layer which diminishes skin friction significantly.

### DETAILED DESCRIPTION OF THE DISCLOSED INVENTION

Silicones are a class of materials with a repeating silicon oxygen or siloxane backbone of few to hundreds of thousands of units. The physical and chemical properties depend on chain length, the identity of the side groups (R₁) and the end groups R₂): 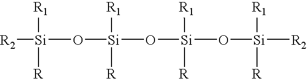
The most widely used and best studied materials use methyl end and side groups (—CH3) and are referred to as poly dimethyl siloxanes. These are also referred to as organopolysilicone materials, because of the combination of carbon and silicon. The most widely used silicone is a linear chain with methyl groups and a low number of repeat units, referred to commonly as dimethicone. Siloxane strands can be form closed ring structures with distinct physical properties, such as the five repeat closed ring cyclopentasiloxane, also referred to as cyclomethicone and as D5, which is volatile. Silicone liquids are oils.

Viscarin (GE) is a trade name for very long chain polydimethylsiloxanes with viscosities from 100,000 to 1,000,000 cP and follow a spectrum from a thick, but flowing liquid to gums which do not flow or deform with change in position. Lubricity results from space filling molecular strands that slide over each other freely.

SFE 818 and SFE 839 (GE) are trade names for combinations of alkyl, polysiloxanes whose side groups permit cross linking so that the molecular threads form a three-dimensional network. Lubricity results from weak cross links that do not sustain shear forces.

Polysilicone-11 is a cross-linked silicone rubber.

These three materials exemplify silicone elastomers with very high viscosity and the physical property of slip or lubricity. Admixing any of these materials with a volatile silicone (i.e. cyclopentasiloxane) or a volatile low molecular weight hydrocarbon (i.e. isododecane) results in a thick, translucent paste which appears to go on dry as the volatile component evaporates, depositing a thin surface layer of the high viscosity component that exhibits sufficient slip and bioadhesiveness to persist on the skin for some hours. This surface layer is gas permeable and water repellant. Bioadhesion is enhanced by adding some silicones with alkyl end groups.

### EXAMPLE I

One preferred formulation is polysilicone-11, 2-10 percent and cyclopentasiloxane 90 to 98 percent. Viscosity is greater than 30,000 cP facilitating application. Since these materials do not support microbial growth, preservatives are not needed, nor is there a risk of hypersensitivity or of skin irritation. Unlike all water soluble gels I have tested, this material does not appear to damage sperm, making it efficacious for sexual relations and for lubricating insertion of diagnostic probes in couples with infertility problems and seeking medical reproductive health assistance.

Oil soluble materials can be added for dermal and transdermal functionality.

Examples include tea tree oil, and oils of rosemary, sage, clary sage, and oregano for their antiseptic and antimicrobial properties, cubeb oil, oils of wintergreen, peppermint, lemon grass, marjoram and menthol and methyl salicylate for their effects on vanilloid nerve endings and sensation, as well as emollients such as squalane, rice bran oil, castor oil, avocado oil, and coconut oil. Antioxidants such as oil soluble forms of vitamins C and E, alpinia leaf oil and other phytoestrogens, may also be added.

In addition, oil soluble substances that appear to increase sexual excitation by an undetermined mechanism may be employed, possibly enhancing the effect of nitric oxide on smooth muscle function, i.e., oils of ginger, pumpkin, and fennel. Any or all of these are mixed typically in concentrations from 0.01 percent to 12 percent, adjusting the balance between the volatile component and the silicone polymer for intended product viscosity. Emulsions can be formed for adding water soluble nitric oxide donor agents, or these can be incorporated into liposomes dispersed throughout the silicone polymer base.

1. A personal lubricant suitable for direct application to the tissues of a user in the absence of application of cosmetic substances for direct dermal and transdermal functionality consisting essentially by weight of from about 2 to 10 percent of a cross-linked silicone rubber and 90 to 98 percent of cyclopentasiloxane to provide a of greater than about 30,000 cp.

2. The lubricant set forth in claim 1, further comprising silicone oil soluble antiseptic and antimicrobial properties.

3. The lubricant set forth in claim 1, further comprising oil soluble materials for stimulating nerve end sensation.
